(19) United States
(12) Patent Application Publication
(10) Pub. No.: US 2006/0251763 A1
(43) Pub. Date: Nov. 9, 2006

(54) Phospholipase variants

(76) Inventors: Shamkant Anant Patkar, Lyngby (DK); Don Higgins, Franklinton, NC (US); Tine Muxoll Fatum, Allerod (DK); Jesper Vind, Vaerlose (DK); Sabry Madkor, Raleigh, NC (US); Thomas Lykke Sorensen, Allerod (DK)

Correspondence Address: NOVOZYMES NORTH AMERICA, INC., 500 FIFTH AVENUE, SUITE 1600, NEW YORK, NY, 10110, US

(21) Appl. No.: 10/561,484

(22) PCT Filed: Jun. 18, 2004
(86) PCT No.: PCT/DK04/00426

§ 371 Date: Dec. 20, 2005

(30) Foreign Application Priority Data
Jun. 19, 2003 (US) 60479647

(51) Int. Cl. A23C 9/12; C12P 21/06; C12N 9/20; C12N 1/21

(52) U.S. Cl. 426/36; 435/69.1; 435/198; 435/320.1; 435/252.3

(57) ABSTRACT

The inventors have used protein engineering to develop variants of fungal phospholipases. Starting from a parent phospholipase, they have modified the amino acid sequence to arrive at variants which have phospholipase activity (generally, at roughly the same level as the parent enzyme) and have a lower lipase activity on triglycerides than the parent enzyme.

### FIELD OF INVENTION

The present invention relates to a method of producing a polypeptide by modifying the amino acid sequence of a polypeptide with phospholipase activity, to a polypeptide having phospholipase activity, and to use of the polypeptide in cheese-making.

### BACKGROUND OF THE INVENTION

Lipolytic enzymes are polypeptides with hydrolytic activity for carboxylic ester bonds, e.g., lipase and/or phospholipase activity. The substrate specificity (relative activity on different ester bonds) is important for the usefulness of the lipolytic enzyme in various industrial applications.

WO 00/32758 discloses lipolytic enzyme variants having altered substrate specificity. WO 98/26057 discloses a Fusarium oxysporum phospholipase. WO 01/83770 describes lipase variants. WO 00/54601 describes a process for producing cheese from cheese milk treated with a phospholipase.

### SUMMARY OF THE INVENTION

The inventors have found that when a fungal phospholipase is used in a cheese-making process, too high lipase activity on triglycerides may lead to a cheese product having changed properties in terms of smell and taste, possibly due to the generation of too many free fatty acids.

To overcome this, the inventors have used protein engineering to develop variants of fungal phospholipases. Starting from a parent phospholipase, they have modified the amino acid sequence to arrive at variants which have phospholipase activity (generally, at roughly the same level as the parent enzyme) and have a lower lipase activity on triglycerides than the parent enzyme. Thus, starting from a parent fungal phospholipase (a polypeptide with phospholipase activity), the inventors have found that the ratio of lipase/phospholipase activity can be decreased by substituting a particular amino acid residue.

The variants are useful in the production of cheese, e.g. in a process or method as described in WO 00/54601, and they result in an increased yield and at the same time avoid the changes in taste and smell, which may result from the generation of too many free fatty acids.

Accordingly, the invention provides a polypeptide which:

a) has phospholipase activity,

b) has an amino acid sequence which is at least 50% identical to SEQ ID NO: 1, and

c) has one or more of the following amino acids at a position corresponding to SEQ ID NO: 1: D62Q/E/F/W/V/P/L/G; V60R/S/K; S85Y/T; G91R/E; R125K; V203T; V228A; T231R; N233R; L259R/V/P; a deletion D266*; and/or L269A.

The invention also provides a method of producing a polypeptide, comprising:

a) selecting a first (parent) polypeptide which has phospholipase activity and has an amino acid sequence which is at least 50% identical to SEQ NO: 1,

b) modifying the amino acid sequence by substituting one or more amino acids at a position corresponding to SEQ ID NO: 1: D62Q/E/F/W/V/P/L/G; V60R/S/K; S85Y/T; G91R/E; V203T; V228A; T231R; N233R; L259R/V/P; a deletion D266*; and/or L269A, and

c) preparing a second (modified) polypeptide having the modified amino acid sequence.

The parent polypeptide may also have lipase activity, and the method may further comprise testing the lipase and phospholipase activities of the two polypeptides and selecting a modified polypeptide having a lower lipase/phospholipase ratio than the parent polypeptide.

Further, the invention provides a polynucleotide encoding the polypeptide and a method for producing cheese, comprising the steps of:

a) treating cheese milk or a fraction of the cheese milk with the polypeptide; and

b) producing cheese from the cheese milk during or after step a).

### BRIEF DESCRIPTION OF DRAWINGS

FIG. 1 shows an alignment of amino acid sequences of known fungal lipolytic enzymes SEQ ID NO: 1 to 14, as follows:

1: Thermomyces lanuginosus (SWISSPROT 059952)

2: Fusarium oxysporum (U.S. Pat. No. 6,103,505 SEQ ID NO: 2, GENESEQP MW51767)

3: Absidia reflexa (U.S. Pat. No. 5,821,102 SEQ ID # 10, GENESEQP MW77403)

4: Absidia corymbifera (U.S. Pat. No. 5,821,102 SEQ ID# 6, GENESEQP MW26689)

5: Rhizomucor miehei (SWISSPROT P19515)

6: Rhizopus oryzae (SWISSPROT P21811)

7: Aspergillus niger (SWISSPROT 042807)

8: Aspergillus tubingensis (SWISSPROT 042815)

9: Fusarium heterosporum (TREMBL Q02351)

10: Aspergillus oryzae (TREMBL P78583)

11: Penicillium camemberti (SWISSPROT P25234)

12: Aspergillus foetidus (U.S. Pat. No. 5,965,422 SEQ ID # 2, GENESEQP MW33009)

13: Aspergillus niger (WO 98/31790 SEQ ID # 2, GENESEQP MW64449)

14: Aspergillus oryzae (JP 10-155493 SEQ ID # 2, GENESEQP AAW 58541)

### DETAILED DESCRIPTION OF THE INVENTION

Parent Polypeptide

The polypeptide of the invention may be derived from a parent polypeptide with phospholipase activity, particularly a phospholipase A1, classified as EC 3.1.1.32 according to Enzyme Nomenclature (available at http://www.chem.qmw.ac.uk/iubmb/enzyme). It may be a naturally occurring fungal enzyme with phospholipase activity, e.g. one of SEQ ID NO: 2-14, particularly a phospholipase from Fusarium oxysporum which is described in WO 98/26057. Alternatively, the parent may be a fungal lipolytic enzyme variant with phospholipase activity as disclosed in WO 00/32758, e.g. a variant of SEQ ID NO: 1 as described in Example 5 of WO 00/32758.

Lipase and Phospholipase Activities

Lipase activity is measured by the SLU method described in WO 0032758, and the lipase activity of the pure protein is expressed as SLU per unit of A280 (Absorption at 280 nm).

Phospholipase activity is measured by incubating 0.025-0.07 mg enzyme protein (e.g. 0.05 mg) with cream (standardized to 25% fat by mixing with skimmed milk) at 35 C for 1.5 hr without shaking and measuring phospholipid depletion (by lipid extraction and HPLC analysis). Phospholipase activity is expressed as % PL depletion.

The variant polypeptides of the invention typically show 15-75% PL depletion by this method. The lipase activity is typically below 1000 SLU/A280, particularly below 500, below 250, below 100 or below 25. The PL/lipase ratio is typically above 0.05, particularly above 0.1, above 0.2, above 0.3, above 1, above 2 or above 3.

The phospholipase activity can also be determined by known methods, e.g. as described in WO 0032758, by HPLC or by phospholipid depletion in cream. Using the “monolayer phospholipase assay” described in WO 0032758, the parent and the modified polypeptide may have a phospholipase activity of at least 0.25 nmol/min at enzyme dose 60 μg and 25° C.; e.g. at least 0.40 nmol/min, at least 0.75 nmol/min, at least 1.0 nmol/min, at least 1.25 nmol/min, or at least 1.5 nmol/min.

Amino Acid Alteration

The modified polypeptide has one or more of the following amino acids at a position corresponding to the following in SEQ ID NO: 1: D62Q/E/F/W/V/P/L/G; V60R/S/K; R84G/S; S85Y/T; G91R/E; R125K; V203T; V228A; T231R; N233R; L259R/V/P; a deletion D266*; and/or L269A. Corresponding positions in SEQ ID NO: 2-14 are defined by the alignment shown in FIG. 1, e.g. position 183 of SEQ ID NO: 2. Corresponding positions in other sequences may be found by an alignment as described below.

Compared to SEQ ID NO: 1, the polypeptide of the invention may further have one or more of the following amino acids at a position corresponding to the following in SEQ ID NO: 1: D57G, V60G/C/K/R/L/S/Q, D62H/A, S83T, R84G/S/W; G91A/V, L93K, D96W/F/G, E99K, R125K, L259S, F262L, G263Q, L264A, I265T, G266D, T267A/E and/or L269N. Also, N- and/or C-terminus may be extended, e.g. as described in WO 9704079. Thus, the C-terminal may be extended by adding residues after position 269, e.g. addition of AGGFS or AGGFSWRRYRSAESVDKRATMTDAELEKKLNSWQMDKEWKNNQARS. The N-terminal may br extended by the addition of amino acid residues such as SPIRR. Such C- or N-terminal extensions should not be considered, when calculating the amino acid identity with SEQ ID NO: 1.

Sequences derived from SEQ ID NO: 2 may be C-terminal processed (e.g. during expression in A. oryzae), e.g. with positions 272, 273, 274 or 286 of SEQ ID NO 2 as the C-terminal residue.

The parent and modified polypeptides may be tested for lipase and phospholipase activity, and a variant polypeptide may be selected which has phospholipase activity and a lipase/phospholipase ratio which is lower than the parent polypeptide. Lipase activity can be determined by known methods using a triglyceride as substrate, e.g. as described in WO 00/32758.

Amino Acid Identity and Alignment

The amino acid identity may be suitably determined by means of computer programs known in the art, such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45), using GAP with the following settings for polypeptide sequence comparison: GAP creation penalty of 3.0 and GAP extension penalty of 0.1.

The variant polypeptide has an amino acid identity to SEQ ID NO: 1 which is at least 50%, particularly at least 55%, at least 60%, at least 65%, at least 70%, at least 75%, at least 80%, at least 85%, at least 90%, at least 95%, or at least 98%.

To find the homologous positions in lipase sequences not shown in the alignment, the sequence of interest is aligned to the sequences shown in FIG. 1. The new sequence is aligned to the present alignment in FIG. 1 by using the GAP alignment to the most homologous sequence found by the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). The following settings are used for polypeptide sequence comparison: GAP creation penalty of 3.0 and GAP extension penalty of 0.1.

### EXAMPLES

### Example 1

### Construction of Variants having a Increased Phospholipase/Lipase Activity Ratio Compared to the Parent Enzyme

The following variant polypeptides were constructed as described in WO 00/32758. Each polypeptide is described by the amino acid alterations compared to SEQ ID NO: 1.

| Variant | Amino acid alteration in SEQ ID NO: 1 |
|---|---|
| 1 | R84W + D96W + E99K + G263Q + L264A + I265T + G266D + T267A + L269N + 270A + |
| | 271G + 272G + 273F + 274S + |
| | 275WRRYRSAESVDKRATMTDAELEKKLNSYVQMDKEYVKNNQARS |
| 2 | R84W + G91E + D96W + E99K + G263Q +L264A + I265T + G266D + T267A + L269N + |
| | 270A + 271G + 272G + 273F + 274S + |
| | 275WRRYRSAESVDKRATMTDAELEKKLNSYVQMDKEYVKNNQARS |
| 3 | V60G + D62E + R84W + G91A + D96F + E99K + G263Q + L264A + I265T + G266D + |
| | T267A + L269N |
| 5 | R84W + G91R + L93K + D96G + E99K + G263Q + L264A + I265T + G266D + T267A + |
| | L269N + 270A + 271G + 272G + 273F + 274S + |
| | 275WRRYRSAESVDKRATMTDAELEKKLNSYVQMDKEYVKNNQARS |
| 6 | V60G + D62F + R84W + G91A + D96W + E99K + G263Q + L264A + I265T + G266D + |
| | T267A + L269N + 270A + 271G + 272G + 273F + 274S + |
| | 275WRRYRSAESVDKRATMTDAELEKKLNSYVQMDKEYVKNNQARS |
| 7 | R84W + S85Y + G91A + D96W + E99K + G263Q + L264A + I265T + G266D + T267A + |
| | L269N + 270A + 271G + 272G + 273F + 274S + |
| | 275WRRYRSAESVDKRATMTDAELEKKLNSYVQMDKEYVKNNQARS |
| 8 | R84W + G91A + D96W + E99K + L259V + G263Q + L264A + I265T + G266D + T267A + |
| | L269N + 270A + 271G + 272G + 273F +274S + |
| | 275WRRYRSAESVDKRATMTDAELEKKLNSYVQMDKEYVKNNQARS |
| 10 | V60G + D62W + R84W + G91A + D96F + E99K + |
| | G263Q + L264A + I265T + G266D + T267A + L269N |
| 11 | R84W + G91R + D96F + E99K + G263Q + L264A + I265T + G266D + T267A + L269N + |
| | 270A + 271G + 272G + 273F + 274S + |
| | 275WRRYRSAESVDKRATMTDAELEKKLNSYVQMDKEYVKNNQARS |
| 12 | V6OC + D62H + R84W + G91A + D96F + E99K + |
| | G263Q + L264A + I265T + G266D + T267A + L269N |
| 13 | V60G + D62V + R84W + G91A + D96F + E99K + |
| | G263Q + L264A + I265T + G266D + T267A + L269N |
| 14 | V60K + D62L + R84W + G91A + D96F + E99K + |
| | G263Q + L264A + I265T + G266D + T267A + L269N |
| 15 | V60R + D62L + R84W + G91A + D96F + E99K + |
| | G263Q + L264A + I265T + G266D + T267A + L269N |
| 16 | V60G + D62G + R84W + G91A + D96W + V228A + E99K + G263Q + L264A + I265T + |
| | G266D + T267A + L269N + 270A + 271G + 272G + 273F + 274S + |
| | 275WRRYRSAESVDKRATMTDAELEKKLNSYVQMDKEYVKNNQARS |
| 17 | V60L + D62A + R84W + G91A + D96W + E99K + R125K + G263Q + L264A + I265T + |
| | G266D + T267A + L269N + 270A + 271G + 272G + 273F + 274S + |
| | 275WRRYRSAESVDKRATMTDAELEKKLNSYVQMDKEYVKNNQARS |
| 18 | D62E + R84W + G91A + D96W + E99K + G263Q + L264A + I265T + G266D + T267A + |
| | L269N + 270A + 271G + 272G + 273F + 274S + |
| | 275WRRYRSAESVDKRATMTDAELEKKLNSYVQMDKEYVKNNQARS |
| 19 | V60S + D62L + R84W + G91A + D96F + E99K + F262L + |
| | G263Q + L264A + I265T + G266D + T267A + L269N |
| 20 | D57G + V60Q + D62P + R84W + G91A + D96F + E99K + |
| | G263Q + L264A + I265T + G266D + T267A + L269N |
| 21 | R84W + G91A + D96W + E99K + L259R + G263Q + L264A + I265T + G266D + T267A + |
| | L269N + 270A + 271G + 272G + 273F + 274S + |
| | 275WRRYRSAESVDKRATMTDAELEKKLNSYVQMDKEYVKNNQARS |
| 23 | D62Q + R84W + G91A + D96W + E99K + G263Q + L264A + I265T + G266D + T267A + |
| | L269N + 270A + 271G + 272G + 273F +274S + |
| | 275WRRYRSAESVDKRATMTDAELEKKLNSYVQMDKEYVKNNQARS |
| 25 | R84W + G91A + D96W + E99K + V203T + G263Q + L264A + I265T + G266D + T267A + |
| | L269N + 270A + 271G + 272G + 273F + 274S + |
| | 275WRRYRSAESVDKRATMTDAELEKKLNSYVQMDKEYVKNNQARS |
| 26 | R84S + S85T + G91A + D96S + T231R + N233R + L259P + |
| | G263Q + L264S + I265T + G266* + T267E + L269A |

Each of the above variant polypeptides showed a phospholipase depletion of 15-75%, a lipase activity below 250 SLU/A280 and a PL/lipase activity above 0.1. For comparison, a number of prior-art variants described in Example 5 of WO 0032758 were measured and were found to have a PL/lipase ratio below 0.05.

### Example 2

### Evaluation of Cheese Yield Using Selected Variants of the Invention

The following variant polypeptides from Example 1 were evaluated in a method of producing cheese with the addition of a phospholipase. The controls were without phospholipase addition.

The method was a bench top cheese yield evaluation test and was performed as described below.

1. Standardize 0.5 kg cheese milk w/ pasteurized skim milk and cream.

2. Prepare a single starter by adding 0.1 g Rhodia LH100 and 0.3 g Rhodia TA061 starter cultures (for mozzarella) to 50 ml of the skim milk and equilibrate to 35° C. w/ gentle, continuous stirring.

3. Equilibrate cheese milk to 35° C. and add 0.07 mg enzyme protein per g fat, check initial pH and add 5 ml starter to each cheese milk with gentle agitation.

4. When pH reaches 6.45-6.50 add 0.5 ml of rennet (10× diluted Chymax, available from Christian Hansen); stir vigorously for three minutes then remove stirrers from milk, cover water bath and allow milk to coagulate.

5. Cut curd at the appropriate time (30-45 minutes) wit 25 mm (½″) knives. To determine cutting time, make a downward cut into the curd with knife or spatula. The curd is ready for cutting when the cut separates upon lifting and sharp edges are maintained on the top surface at the edge of the cut. Allow the curd to rest for 5 minutes then gently and intermittently stir curd to prevent coalescence of curd particles.

6. Increase temperature to 41° C. and hold until curd pH reaches 5.65-5.70, then drain and pour curd particles into stainless steel bowls. Float bowls in 41° C. water bath to maintain curd temperature. Periodically drain excess whey, leaving only enough to cover curds for maintenance of heat.

7. When curd pH ˜5.25-5.3, drain all whey and flood curd w/ D.I. water at 57° C. for 5 min. Stretch the curd by hand for ˜1 min in 59° C. water, then place the curd in ice water for 15 min and dry blot. Record weight of curd and refrigerate until further analysis.

Results

Variants No. 2, 4, 5, 8, 9, 10, 16, 22 and 24 of Example 1 were tested. All the tested variants resulted in improved yield compared to the control, when calculated as moisture adjusted yield.

1-7. (canceled)

8. A polypeptide which:
a) has phospholipase activity,
a) has an amino acid sequence which is at least 50% identical to SEQ ID NO: 1, and
b) has one or more of the following amino acid alterations: D62Q/E/F/W/V/P/L/G; V60R/S/K; S85Y/T; G91R/E; R125K; V203T; V228A; T231R; N233R; L259R/V/P; a deletion D266*; and/or L269A (using SEQ ID NO:1 for numbering).

9. The polypeptide of claim 8, which has one or more of the following amino acids alterations D57G, V60G/C/L/Q, D62H/A, S83T, R84G/S/W; G91A/V, L93K, D96W/F/G, E99K, R125K, L259S, F262L, G263Q, L264A, I265T, G266D, T267A/E, L269N and/or by a C-terminal extension.

10. The polypeptide of claim 9, wherein the C-terminal extension is AGGFS or AGGFSWRRYRSAESVDKRATMTDAELEKKLNSYVQMDKEYVKNNQARS.

11. The polypeptide of claim 8, which has the sequence of SEQ ID NO: 1 with one of the following sets of alterations:
R84W + D96W + E99K + G263Q + L264A + I265T + G266D +
T267A + L269N + 270A + 271G + 272G + 273F + 274S +
275WRRYRSAESVDKRATMTDAELEKKLNSYVQMDKEYVKNNQARS
R84W + G91E + D96W + E99K + G263Q + L264A + I265T +
G266D + T267A + L269N + 270A + 271G + 272G + 273F + 274S +
275WRRYRSAESVDKRATMTDAELEKKLNSYVQMDKEYVKNNQARS
V60G + D62E + R84W + G91A + D96F + E99K + G263Q +
L264A + I265T + G266D + T267A + L269N
R84W + G91R + L93K + D96G + E99K + G263Q + L264A + I265T +
G266D + T267A + L269N + 270A + 271G + 272G + 273F + 274S +
275WRRYRSAESVDKRATMTDAELEKKLNSYVQMDKEYVKNNQARS
V60G + D62F + R84W + G91A + D96W + E99K + G263Q + L264A +
I265T + G266D + T267A + L269N + 270A + 271G + 272G + 273F + 274S +
275WRRYRSAESVDKRATMTDAELEKKLNSYVQMDKEYVKNNQARS
R84W + S85Y + G91A + D96W + E99K + G263Q + L264A + I265T +
G266D + T267A + L269N + 270A + 271G + 272G + 273F + 274S +
275WRRYRSAESVDKRATMTDAELEKKLNSYVQMDKEYVKNNQARS
R84W + G91A + D96W + E99K + L259V + G263Q + L264A + I265T +
G266D + T267A + L269N + 270A + 271G + 272G + 273F + 274S +
275WRRYRSAESVDKRATMTDAELEKKLNSYVQMDKEYVKNNQARS
V60G + D62W + R84W + G91A + D96F + E99K + G263Q +
L264A + I265T + G266D + T267A + L269N
R84W + G91R + D96F + E99K + G263Q + L264A + I265T +
G266D + T267A + L269N + 270A + 271G + 272G + 273F + 274S +
275WRRYRSAESVDKRATMTDAELEKKLNSYVQMDKEYVKNNQARS
V6OC + D62H + R84W + G91A + D96F + E99K + G263Q +
L264A + I265T + G266D + T267A + L269N
V60G + D62V + R84W + G91A + D96F + E99K + G263Q +
L264A + I265T + G266D + T267A + L269N
V60K + D62L + R84W + G91A + D96F + E99K + G263Q +
L264A + I265T + G266D + T267A + L269N
V60R + D62L + R84W + G91A + D96F + E99K + G263Q +
L264A + I265T + G266D + T267A + L269N
V60G + D62G + R84W + G91A + D96W + V228A + E99K +
G263Q + L264A + I265T + G266D + T267A + L269N +
270A + 271G + 272G + 273F + 274S +
275WRRYRSAESVDKRATMTDAELEKKLNSYVQMDKEYVKNNQARS
V60L + D62A + R84W + G91A + D96W + E99K + R125K +
G263Q + L264A + I265T + G266D + T267A + L269N +
270A + 271G + 272G + 273F + 274S +
275WRRYRSAESVDKRATMTDAELEKKLNSYVQMDKEYVKNNQARS
D62E + R84W + G91A + D96W + E99K + G263Q +
L264A + I265T + G266D + T267A + L269N +
270A + 271G + 272G + 273F + 274S +
275WRRYRSAESVDKRATMTDAELEKKLNSYVQMDKEYVKNNQARS
V60S + D62L + R84W + G91A + D96F + E99K + F262L +
G263Q + L264A + I265T + G266D + T267A + L269N
D57G + V60Q + D62P + R84W + G91A + D96F + E99K +
G263Q + L264A + I265T + G266D + T267A + L269N
R84W + G91A + D96W + E99K + L259R + G263Q +
L264A + I265T + G266D + T267A + L269N +
270A + 271G + 272G + 273F + 274S +
275WRRYRSAESVDKRATMTDAELEKKLNSYVQMDKEYVKNNQARS
D62Q + R84W + G91A + D96W + E99K + G263Q +
L264A + I265T + G266D + T267A + L269N +
270A + 271G + 272G + 273F + 274S +
275WRRYRSAESVDKRATMTDAELEKKLNSYVQMDKEYVKNNQARS
R84W + G91A + D96W + E99K + V203T + G263Q +
L264A + I265T + G266D + T267A + L269N +
270A + 271G + 272G + 273F + 274S +
275WRRYRSAESVDKRATMTDAELEKKLNSYVQMDKEYVKNNQARS
R84S + S85T + G91A + D96S + T231R + N233R +
L259P + G263Q + L264S + I265T + G266* + T267E + L269A

12. The polypeptide or claim 8, which has an amino acid sequence which is at least 60% identical to SEQ ID NO: 1.

13. The polypeptide or claim 8, which has an amino acid sequence which is at least 70% identical to SEQ ID NO: 1.

14. The polypeptide or claim 8, which has an amino acid sequence which is at least 80% identical to SEQ ID NO: 1.

15. The polypeptide or claim 8, which has an amino acid sequence which is at least 90% identical to SEQ ID NO: 1.

16. The polypeptide or claim 8, which has an amino acid sequence which is at least 95% identical to SEQ ID NO: 1.

17. The polypeptide or claim 8, which has an amino acid sequence which is at least 98% identical to SEQ ID NO: 1.

18. A polynucleotide encoding the polypeptide of claim 8.

19. A method of producing a polypeptide, comprising:
a) selecting a first polypeptide which has phospholipase activity and has an amino acid sequence which is at least 50% identical to SEQ NO: 1,
b) altering the amino acid sequence wherein the alteration comprises one or more substitutions or deletion corresponding to the following in SEQ ID NO: 1: D62Q/E/F/W/V/P/L/G; V60R/S/K; S85Y/T; G91 R/E; V203T; V228A; T231R; N233R; L259R/V/P; a deletion D266*; and/or L269A.

20. A method for producing cheese, comprising the steps of:
a) treating cheese milk or a fraction of the cheese milk with the polypeptide claim 8; and
b) producing cheese from the cheese milk during or after step a).


SEQUENCE LISTING (14 sequences)

SEQ ID NO: 1 - protein, 269 aa, Thermomyces lanuginosus [sequence omitted]

SEQ ID NO: 2 - protein, 286 aa, Fusarium oxysporum
AVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKR

SEQ ID NO: 3 - protein, 265 aa, Absidia reflexa
SSSSTQDYRIASEAEIKAHTFYTALSANAYCRTVIPGGRWSCPHCGVASNLQITKTFSTLITDTNVLVAVGEKEKTIYVVFRGTSSIRNAIADIVFVPVNYPPVNGAKVHKGFLDSYNEVQDKLVAEVKAQLDRHPGYKIVVTGHSLGGATAVLSALDLYHHGHANIEIYTQGQPRIGTPAFANYVIGTKIPYQRLVHERDIVPHLPPGAFGFLHAGEEFWIMKDSSLRVCPNGIETDNCSNSIVPFTSVIDHLSYLDMNTGLCL

SEQ ID NO: 4 - protein, 264 aa, Absidia corymbifera
SSSTQDYRIASEAEIKAHTFYTALSANAYCRTVIPGGQWSCPHCDVAPNLNITKTFTTLITDTNVLVAVGENEKTIYVVFRGTSSIRNAIADIVFVPVNYPPVNGAKVHKGFLDSYNEVQDKLVAEVKAQLDRHPGYKIVVTGHSLGGATAVLSALDLYHHGHDNIEIYTQGQPRIGTPEFANYVIGTKIPYQRLVNERDIVPHLPPGAFGFLHAGEEFWIMKDSSLRVCPNGIETDNCSNSIVPFTSVIDHLSYLDMNTGLCL

SEQ ID NO: 5 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 6 - protein, 271 aa, Rhizopus oryzae
SASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 7 - protein, 267 aa, Aspergillus niger
TAGQALAASTQGISEDLYNRLVEMATISQAAYADLCNIPSTIIKGEKIYNAQTDINGWILRDDTSKEIITVFRGTGSDTNLQLDTNYTLTPFDTLPQCNDCEVHGGYYIGWISVQDQVESLVKQQASQYPDYALTVTGHSLGASMAALTAAQLSATYDNVRLYTFGEPRSGNQAFASYMNDAFQVSSPETTQYFRVTHSNDGIPNLPPADEGYAHGGVEYWSVDPYSAQNTFVCTGDEVQCCEAQGGQGVNDAHTTYFGMTSGACTW

SEQ ID NO: 8 - protein, 266 aa, Aspergillus tubingensis
TAGHALAASTQGISEDLYSRLVEMATISQAAYADLCNIPSTIIKGEKIYNSQTDINGWILRDDSSKEIITVFRGTGSDTNLQLDTNYTLTPFDTLPQCNSCEVHGGYYIGWISVQDQVESLVQQQVSQFPDYALTVTGHSLGASLAALTAAQLSATYDNIRLYTFGEPRSNQAFASYMNDAFQASSPDTTQYFRVTHANDGIPNLPPADEGYAHGVVEYWSVDPYSAQNTFVCTGDEVQCCEAQGGQGVNNAHTTYFGMTSGHCTW

SEQ ID NO: 9 - protein, 273 aa, Fusarium heterosporum
TVTTQDLSNFRFYLQHADAAYCNFNTAVGKPVHCSAGNCPDIEKDAAIVVGSVVGTKTGIGAYVATDNARKEIVVSVRGSINVRNWITNFNFGQKTCDLVAGCGVHTGFLDAWEEVAANVKAAVSAAKTANPTFKFVVTGHSLGGAVATIAAAYLRKDGFPFDLYTYGSPRVGNDFFANFVTQQTGAEYRVTHGDDPVPRLPPIVFGYRHTSPEYWLNGGPLDKDYTVTEIKVCEGIANVMCNGGTIGLDILAHITYFQSMATCAPIAIPWKR

SEQ ID NO: 10 - protein, 278 aa, Aspergillus oryzae
DIPTTQLEDFKFWVQYAAATYCPNNYVAKDGEKLNCSVGNCPDVEAAGSTVKLSFSDDTITDTAGFVAVDNTNKAIVVAFRGSYSIRNWVTDATFPQTDPGLCDGCKAELGFWTAWKVVRDRIIKTLDELKPEHSDYKIVVVGHSLGAAIASLAAADLRTKNYDAILYAYAAPRVANKPLAEFITNQGNNYRFTHNDDPVPKLPLLTMGYVHISPEYYITAPDNTTVTDNQVTVLDGYVNFKGNTGTSGGLPDLLAFHSHVWYFIHADACKGPGLPLR

SEQ ID NO: 11 - protein, 278 aa, Penicillium camemberti
DVSTSELDQFEFWVQYAAASYYEADYTAQVGDKLSCSKGNCPEVEATGATVSYDFSDSTITDTAGYIAVDHTNSAVVLAFRGSYSVRNWVADATFVHTNPGLCDGCLAELGFWSSWKLVRDDIIKELKEVVAQNPNYELVVVGHSLGAAVATLAATDLRGKGYPSAKLYAYASPRVGNAALAKYITAQGNNFRFTHTNDPVPKLPLLSMGYVHVSPEYWITSPNNATVSTSDIKVIDGDVSFDGNTGTGLPLLTDFEAHIWYFVQVDAGKGPGLPFKR

SEQ ID NO: 12 - protein, 270 aa, Aspergillus foetidus
SVSTSTLDELQLFAQWSAAAYCSNNIDSKDSNLTCTANACPSVEEASTTMLLEFDLTNDFGGTAGFLAADNTNKRLVVAFRGSSTIENWIANLDFILEDNDDLCTGCKVHTGFWKAWESAADELTSKIKSAMSTYSGYTLYFTGHSLGGALATLGATVLRNDGYSVELYTYGCPRIGNYALAEHITSQGSGANFRVTHLNDIVPRVPPMDFGFSQPSPEYWITSGNGASVTASDIEVIEGINSTAGNAGEATVSVLAHLWYFFAISECLL

SEQ ID NO: 13 - protein, 270 aa, Aspergillus niger
SVSTSTLDELQLFSQWSAAAYCSNNIDSDDSNVTCTADACPSVEEASTKMLLEFDLTNNFGGTAGFLAADNTNKRLVVAFRGSSTIKNWIADLDFILQDNDDLCTGCKVHTGFWKAWEAAADNLTSKIKSAMSTYSGYTLYFTGHSLGGALATLGATVLRNDGYSVELYTYGCPRVGNYALAEHITSQGSGANFPVTHLNDIVPRVPPMDFGFSQPSPEYWITSGTGASVTASDIELIEGINSTAGNAGEATVDVLAHLWYFFAISECLL

SEQ ID NO: 14 - protein, 269 aa, Aspergillus oryzae
DVSSSLLNNLDLFAQYSAAAYCDENLNSTGTKLTCSVGNCPLVEAASTQSLDEFNESSSYGNPAGYLAADETNKLLVLSFRGSADLANWVANLNFGLEDASDLCSGCEVHSGFWKAWSEIADTITSKVESALSDHSDYSLVLTGHSYGAALAALAATALRNSGHSVELYNYGQPRLGNEALATYITDQNKGGNYRVTHTNDIVPKLPPTLLGYHHFSPEYYISSADEATVTTTDVTEVTGIDATGGNDGTDGTSIDAHRWYFIYISECS